(19) United States
(12) Patent Application Publication
(10) Pub. No.: US 2001/0049378 A1
(43) Pub. Date: Dec. 6, 2001

(54) METHODS OF USE OF FLUOROQUINOLONE COMPOUNDS AGAINST MAXILLARY SINUS PATHOGENIC BACTERIA

(76) Inventors: JACQUES DUBOIS, FLEURAMONT (CA); CLAUDE ST. PIERRE, ST. ELIE D'ORFORD (CA)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 09/395,492

(22) Filed: Sep. 14, 1999

Related U.S. Application Data
(60) Provisional application No. 60/141,486, filed on Jun. 29, 1999.

(51) Int. Cl. A61K 31/47; A01N 43/42; A61K 31/44; A01N 43/40

(52) U.S. Cl. 514/312

(57) ABSTRACT

This invention relates, in part, to newly identified methods of using quinolone antibiotics, particularly a gemifloxacin compound against maxillary sinus pathogenic pathogenic bacteria

This invention relates, in part, to newly identified methods of using quinolone antibiotics, particularly a gemifloxacin compound against maxillary sinus pathogenic pathogenic bacteria, such as penicillin-resistant and ciprofloxacin-resistant bacteria, especially resistant Streptococcus pneumontae.

### BACKGROUND OF THE INVENTION

Quinolones have been shown to be effective to varying degrees against a range of bacterial pathogens. However, as diseases caused by these pathogens are on the rise, there exists a need for antimicrobial compounds that are more potent than the present group of quinolones.

Gemifloxacin mesylate (SB-265805) is a novel fluoroquinolone useful as a potent antibacterial agent. Gemifloxacin compounds are described in detail in patent application PCT/KR98/00051 published as WO 98/42705. Patent application EP 688772 discloses novel quinoline(naphthyridine)carboxylic acid derivatives, including anhydrous (R,S)-7-(3-aminomethyl-4-methoxyiminopyrrolidin-1-yl)-1-cyclopropyl-6-fluoro-4-oxo-1,4-dihydro-1,8-naphthyridine-3-carboxylic acid of formula I.

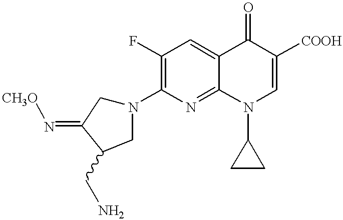

PCT/KR98/00051 discloses (R,S)-7-(3-aminomethyl-4-syn-methoxyimino-pyrrolidin-1-yl)-1-cyclopropyl-6-fluoro-4-oxo-1,4-dihydro-1,8-naphthyridine-3-carboxylic acid methanesulfonate and hydrates thereof including the sesquihydrate.

Provided herein is a significant discovery made using a gemifloxacin compound against a range of respiratory pathogens, demonstrating the activity of the gemifloxacin compound used was superior to a number of quinolones as described in more detail herein. Gemifloxacin compounds are valuable compounds for the treatment of acute or chronic sinusitis caused by a range of respiratory pathogens, including those resistant to usual oral therapy, thereby filling an unmet medical need.

### SUMMARY OF THE INVENTION

An object of the invention is a method for modulating metabolism of maxillary sinus pathogenic bacteria comprising the step of contacting maxillary sinus pathogenic bacteria with an antibacterially effective amount of a composition comprising a gemifloxacin compound, or an antibacterially effective derivative thereof.

A further object of the invention is a method wherein said maxillary sinus pathogenic bacteria is selected from the group consisting of: a bacterial strains isolated from acute or chronic maxillary sinusitis; and a maxillary sinus isolate of S. aureus, S. pneumoniae, Haemophilus spp., M. catarrhalis, and anaerobic strain or non-fermentative Gram negative bacilli, Neisseria meningitidis and β-haemolytic Streptococcus.

Also provided by the invention is a method of treating or preventing a bacterial infection by maxillary sinus pathogenic bacteria comprising the step of administering an antibacterially effective amount of a composition comprising a gemifloxacin compound to a mammal suspected of having or being at risk of having an infection with maxillary sinus pathogenic bacteria.

A preferred method is provided wherein said modulating metabolism is inhibiting growth of said bacteria or killing said bacteria.

A further preferred method is provided wherein said contacting said bacteria comprises the further step of introducing said composition into a mammal, particularly a human.

Further preferred methods are provided by the invention wherein said bacteria is selected from the group consisting of: a bacterial strain isolated from acute or chronic maxillary sinusitis; a maxillary sinus isolate of Staphylococcus aureus, Streptococcus pneumoniae, Haemophilus spp., Moraxella catarrhalis, an anaerobic strain or non-fermentative Gram negative bacilli, Neisseria meningitidis, β-haemolytic Streptococcus, Haemophilus influenzae, an Enterobacteriaceae, a non-fermentative Gram negative bacilli, Streptococcus pneumoniae, Streptococcus pyogenes, a methicillin-resistant Staphylococcus spp., Legionella pneumophila, Mycoplasma spp. and Chlamydia spp., Haemophilus influenzae, Haemophilus parainfluenzae, Peptostreptococcus, Bacteroides spp., and Bacteroides urealyticus.

Various changes and modifications within the spirit and scope of the disclosed invention will become readily apparent to those skilled in the art from reading the following descriptions and from reading the other parts of the present disclosure.

### DESCRIPTION OF THE INVENTION

The present invention provides, among other things, methods for using a composition comprising a gemifloxacin compound against maxillary sinus pathogenic bacteria, especially maxillary sinus strains of S. aureus, S. pneumoniae, Haemophilus spp., M. catarrhalis, certain anaerobic strains, non-fermentative Gram negative bacilli, Neisseria meningitidis and β-haemolytic Streptococcus.

As used herein “gemifloxacin compound(s)” means a compound having antibacterial activity described in patent application PCT/KR98/00051 published as WO 98/42705, or patent application EP 688772.

This invention was based, in part, on analyses evaluating the comparative activity of gemifloxacin against various maxillary sinus pathogens. An objective of these analyses was to determine minimum inhibitory concentrations (herein “MIC”) of gemifloxacin, ciprofloxacin, ofloxacin, levofloxacin, trovafloxacin, grepafloxacin, moxifloxacin, sparfloxacin, amoxycillin and amoxycillin/clavulanic acid against a variety of strains such as Haemophilus spp. S. pneumoniae and Moraxella catarrhalis, isolated recently from acute or chronic maxillary sinus infections.

Gemifloxacin was compared to ciprofloxacin, ofloxacin, levofloxacin, trovafloxacin, grepafloxacin, moxifloxacin, sparfloxacin, amoxycillin and amoxycillin/clavulanic acid against a total of more than 250 recent isolates from acute or chronic maxillary sinusitis. MICs were determined by agar dilution techniques using standard NCCLS methodology. The activity of gemifloxacin (MIC₉₀ 0.06 mg/L) was superior to ciprofloxacin, ofloxacin, levofloxacin, grepafloxacin, moxifloxacin and sparfloxacin (MIC₉₀≧0.25 mg/L) against the Streptococcus pneumoniae isolates tested. Against Moraxella catarrhalis and Haemophilus influenzae, gemifloxacin and grepafloxacin (MIC⁹⁰≧0.02 mg\L) were the most active antimicrobial agents tested. Against Staphylococcus aureus, gemifloxacin, trovafloxacin and moxifloxacin were more active (MIC₉₀ 0.06 mg\L) than ciprofloxacin amoxycillin and amoxycillin/clavulanic acid (MIC₉₀≧1 mg\L). A similar activity (MIC₉₀ 0.25 mg\L) was observed with gemifloxacin and moxifloxacin against anaerobic strains tested. The activity of gemifloxacin was similar to ofloxacin, trovafloxacin, moxifloxacin and sparfloxacin (MIC₉₀ 0.5 mg/L) against various other strains such as some Enterobacteriaceae or non-fermentative Gram negative bacilli. Combined with favourable pharmacokinetics in humans, gemifloxacin should be a valuable oral compound for the treatment of acute or chronic sinusitis caused by a range of respiratory pathogens, including those resistant to usual oral therapy. The susceptibility results are presented in Tables 2-5.

These analyses showed that gemifloxacin is appreciably more potent than most fluoroquinolones against many Gram positive organisms, including Streptococcus pneumoniae, Streptococcus pyogenes and methicillin-resistant Staphylococcus spp. Gemifloxacin retains activity against a range of Gram negative bacilli, including those resistant to other antimicrobial agents. It also has potent activity against various anaerobic and atypical respiratory pathogens, such as Legionella pneumophila, Mycoplasma spp. and Chlamydia spp.

Against S. pneumoniae, gemifloxacin activity (MIC₉₀ 0.06 mg/L) was similar to trovafloxacin, but superior to ciprofloxacin, ofloxacin, levofloxacin and sparfloxacin (MIC₉₀<0.5 mg/L) (Table 2). Against S. aureus sinus pathogens, gemifloxacin, moxifloxacin, trovafloxacin (MIC₉₀ 0.06 mg/L) and sparfloxacin (MIC₉₀ 0.12 mg/L) were the most active compounds tested. Ciprofloxacin, amoxycillin (MIC₉₀ 1 mg/L) and amoxycillin/clavulanic acid (MIC₉₀ 2 mg/L) were less active against S. aureus (Table 2).

H. influenzae strains were susceptible to gemifloxacin at a MIC₉₀ of ≦0.02 mg/L (Table 3). This activity was significantly superior to ofloxacin, moxifloxacin, sparfloxacin, amoxycillin and amoxycillin/clavulanic acid. Against Haemophilus parainfluenzae, gemifloxacin (MIC₉₀ 0.12 mg/L) was superior to ofloxacin (MIC₉₀ 0.5 mg/L), moxifloxacin (MIC₉₀ 0.5 mg/L), sparfloxacin (MIC₉₀ 1 mg/L), amoxycillin (MIC₉₀ 1 mg/L) and amoxycillin/clavulanic acid (MIC₉₀ 0.5 mg/L).

Against M. catarrhalis, gemifloxacin and grepafloxacin (MIC₉₀≦0.02 mg/L) were the most active compounds tested (Table 4). Gemifloxacin was significantly more potent than sparfloxacin, amoxycillin/clavulanic acid (MIC₉₀ 0.5 mg/L) and amoxycillin (MIC₉₀ 8 mg/L).

Against anaerobic strains, gemifloxacin (MIC₉₀ 0.25 mg/L) and moxifloxacin (MIC₉₀ 0.25 mg/L) were the most active agents tested (Table 5). The activity of gemifloxacin was significantly superior to ofloxacin (MIC₉₀ 2 mg/L), trovafloxacin (MIC₉₀ 4 mg/L), grepafloxacin (MIC₉₀ 8 mg/L) and sparfloxacin (MIC₉₀ 16 mg/L). Against various other streptococcal strains, gemifloxacin was as active as ofloxacin, trovafloxacin, moxifloxacin and sparfloxacin (MIC₉₀ 0.5 mg/L).

Gemifloxacin shows a broad spectrum of antibacterial activity against a broad range of bacterial strains isolated from acute or chronic maxillary sinusitis.

The activity of gemifloxacin was higher than other agents tested against a broad range of maxillary sinus isolates, such as, for example, S. aureus, Haemophilus spp., M. catarrhalis and anaerobic strains. The overall in vitro activity of this compound is significantly greater than ciprofloxacin, ofloxacin, levofloxacin and sparfloxacin against strains of S. pneumoniae. Gemifloxacin also has significant activity against Haemophilus spp., M. catarrhalis, some anaerobic strains and other various strains tested such as: non-fermentative Gram negative bacilli, Neisseria meningitidis and β-haemolytic Streptococcus. Combined with favourable pharmacokinetics in humans, gemifloxacin is a valuable oral compound for the treatment of acute or chronic sinusitis caused by microbial agents resistant to usual oral therapy.

TABLE 1
Test Strains Isolated from Maxillary Sinus Pathogens

| Microrganism | No. of tested strains |
|---|---|
| Streptococcus pneumoniae | 85 |
| Haemophilus influenzae | 45 |
| Haemophilus parainfluenzae | 10 |
| Moraxella catarrhalis | 45 |
| Staphylococcus aureus | 31 |
| Anaerobes* | 22 |
| Other spp.^{†} | 15 |

*Including Peptostreptococcus and Bacteroides spp.
^{†}Including beta-haemolytic Streptococcus and Gram negative rods

TABLE 2
Susceptibility of Gram Positive Cocci

| | S. pneumoniae (n = 85) | | | S. aureus (n = 31) | | |
|---|---|---|---|---|---|---|
| Anti- | MIC (mg/L) | | | MIC (mg/L) | | |
| microbial | Range | 50% | 90% | Range | 50% | 90% |
| Gemi- | ≦0.02-0.06 | 0.03 | 0.06 | 0.03-1 | 0.06 | 0.06 |
| floxacin | | | | | | |
| Moxi- | ≦0.02-0.25 | 0.12 | 0.25 | 0.03-0.12 | 0.06 | 0.06 |
| floxacin | | | | | | |
| Trova- | ≦0.02-0.12 | 0.06 | 0.12 | ≦0.02-0.06 | 0.03 | 0.03 |
| floxacin | | | | | | |
| Grepa- | 0.03-0.5 | 0.25 | 0.25 | 0.06-0.25 | 0.12 | 0.12 |
| floxacin | | | | | | |
| Levofloxacin | 0.12-2 | 1 | 1 | 0.12-0.5 | 0.25 | 0.25 |
| Ofloxacin | 0.25-4 | 2 | 2 | 0.25-1 | 0.5 | 0.5 |
| Sparfloxacin | 0.03-0.5 | 0.25 | 0.5 | 0.3-0.12 | 0.06 | 0.12 |
| Cipro- | 0.06-2 | 0.5 | 1 | 0.12-1 | 0.5 | 1 |
| floxacin | | | | | | |
| Amoxycillin | ≦0.02-1 | 0.03 | 0.03 | 0.06-2 | 1 | 1 |
| Amox/clav | ≦0.02-1 | ≦0.02 | 0.03 | 0.03-2 | 1 | 1 |

TABLE 3
Susceptibility of Haemophilus spp.

| | H. influenzae (n = 45) | | | H. parainfluenzae (n = 10) | | |
|---|---|---|---|---|---|---|
| Anti- | MIC (mg/L) | | | MIC (mg/L) | | |
| microbial | Range | 50% | 90% | Range | 50% | 90% |
| Gemi- | ≦0.02-0.03 | ≦0.02 | ≦0.02 | ≦0.02-0.12 | 0.06 | 0.12 |
| floxacin | | | | | | |
| Moxi- | ≦0.02-0.12 | 0.13 | 0.06 | 0.06-0.5 | 0.25 | 0.5 |
| floxacin | | | | | | |
| Trova- | ≦0.02-0.06 | ≦0.02 | 0.03 | ≦0.02-0.12 | 0.03 | 0.12 |
| floxacin | | | | | | |
| Grepa- | ≦0.02-0.03 | ≦0.02 | ≦0.02 | ≦0.02-0.12 | 0.06 | 0.1 |
| floxacin | | | | | | |
| Levo- | ≦0.02-0.03 | 0.03 | 0.03 | 0.03-0.25 | 0.06 | 0.25 |
| floxacin | | | | | | |
| Ofloxacin | ≦0.02-0.06 | 0.03 | 0.06 | 0.03-0.5 | 0.12 | 0.5 |
| Spar- | 0.03-1 | 0.25 | 0.25 | 0.12-1 | 0.5 | 1 |
| floxacin | | | | | | |
| Cipro- | ≦0.02 | ≦0.02 | ≦0.02 | ≦0.02-0.06 | 0.03 | 0.06 |
| floxacin | | | | | | |
| Amoxy- | 0.06-64 | 0.25 | 2 | 0.03-1 | 0.06 | 1 |
| cillin | | | | | | |
| Amox/ | ≦0.02-1 | 0.25 | 0.5 | 0.03-0.5 | 0.25 | 0.5 |
| clav | | | | | | |

TABLE 4
Susceptibility of Moraxella catarrhalis

| | M. catarrhalis (n = 45) | | |
|---|---|---|---|
| | MIC (mg/L) | | |
| Antimicrobial | Range | 50% | 90% |
| Gemifloxacin | ≦0.02-0.03 | ≦0.02 | ≦0.02 |
| Moxifloxacin | 0.03-0.12 | 0.06 | 0.06 |
| Trovafloxacin | ≦0.02-0.06 | ≦0.02 | 0.03 |
| Grepafloxacin | ≦0.02-0.25 | ≦0.02 | ≦0.02 |
| Levofloxacin | ≦0.02-0.12 | 0.03 | 0.06 |
| Ofloxacin | ≦0.02-0.25 | 0.06 | 0.06 |
| Sparfloxacin | ≦0.02-1 | ≦0.02 | 0.5 |
| Ciprofloxacin | ≦0.02-0.25 | 0.03 | 0.03 |
| Amoxycillin | ≦0.02-16 | 1 | 8 |
| Amox/clav | ≦0.02-2 | 0.12 | 0.5 |

TABLE 5
Susceptibility of Anaerobic and Streptococcal Strains

| | Anaerobic strains (n = 22)* | | | Streptococcus spp.^{†} | | |
|---|---|---|---|---|---|---|
| | MIC (mg/L) | | | MIC (mg/L) | | |
| Antimicrobial | Range | 50% | 90% | Range | 50% | 90% |
| Gemifloxacin | 0.03-0.25 | 0.12 | 0.25 | ≦0.02-0.5 | 0.12 | 0.5 |
| Moxifloxacin | 0.03-0.25 | 0.03 | 0.25 | ≦0.02-0.5 | 0.06 | 0.5 |
| Trovafloxacin | 0.06-4 | 1 | 4 | ≦0.02-0.5 | 0.06 | 0.5 |
| Grepafloxacin | 0.25-8 | 0.25 | 8 | ≦0.02-1 | 0.06 | 1 |
| Levofloxacin | 0.12-1 | 0.25 | 1 | 0.03-0.25 | 0.12 | 0.25 |
| Ofloxacin | 0.25-2 | 0.5 | 2 | 0.06-0.5 | 0.25 | 0.5 |
| Sparfloxacin | 0.25-16 | 4 | 16 | ≦0.02-0.5 | 0.03 | 0.5 |
| Ciprofloxacin | 0.06-1 | 0.5 | 1 | ≦0.02-0.12 | 0.12 | 0.12 |
| Amoxycillin | 0.25-8 | 0.25 | 8 | 0.03-≧256 | 2 | 4 |
| Amox/clav | 0.25-1 | 0.25 | 1 | 0.03-≧256 | 2 | 16 |

*Including 12 strains of Bacteroides spp., 7 strains of Peptostreptococcus spp. and 3 strains of Bacteroides urealyticus.
^{†}Including 5 strains of Enterobacteriaceae, 6 strains of non-fermentative Gram negative bacilli, 2 strains of Neisseria meningitidis and 2 strains of beta-haemolytic Streptococcus.

The invention provides a method for modulating metabolism of maxillary sinus pathogenic bacteria. Skilled artisans can readily choose maxillary sinus pathogenic bacteria or patients infected with or suspected to be infected with these organisms to practice the methods of the invention. Alternatively, the bacteria useful in the methods of the invention may be those described herein.

The contacting step in any of the methods of the invention may be performed in many ways that will be readily apparent to the skilled artisan. However, it is preferred that the contacting step is a provision of a composition comprising a gemifloxacin compound to a human patient in need of such composition or directly to bacteria in culture medium or buffer.

For example, when contacting a human patient or contacting said bacteria in a human patient or in vitro, the compositions comprising a gemifloxacin compound, preferably pharmaceutical compositions may be administered in any effective, convenient manner including, for instance, administration by topical, oral, anal, vaginal, intravenous, intraperitoneal, intramuscular, subcutaneous, intranasal or intradermal routes among others.

It is also preferred that these compositions be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a compound of the invention, preferably a gemifloxacin compound, and a pharmaceutically acceptable carrier or excipient. Such carriers may include, but are not limited to, saline, buffered saline, dextrose, water, glycerol, ethanol and combinations thereof. The formulation should suit the mode of administration.

Gemifloxacin compounds and compositions of the methods of the invention may be employed alone or in conjunction with other compounds, such as bacterial efflux pump inhibitor compounds or antibiotic compounds, particularly non-quinolone compounds, e.g., beta-lactam antibiotic compounds.

In therapy or as a prophylactic, the active agent of a method of the invention is preferably administered to an individual as an injectable composition, for example as a sterile aqueous dispersion, preferably an isotonic one.

Alternatively, the gemifloxacin compounds or compositions in the methods of the invention may be formulated for topical application for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the antibacterially effective amount is a daily dosage level of the active agent from 0.001 mg/kg to 10 mg/kg, typically around 0.1 mg/kg to 1 mg/kg, preferably about 1 mg/kg. A physician, in any event, will determine an actual dosage that is most suitable for an individual and will vary with the age, weight and response of the particular individual. The above dosages are exemplary of the average case. There can, of course, be individual instances where higher or lower dosage ranges are merited, and such are within the scope of this invention. It is preferred that the dosage is selected to modulate metabolism of the bacteria in such a way as to inhibit or stop growth of said bacteria or by killing said bacteria. The skilled artisan may identify this amount as provided herein as well as using other methods known in the art, e.g. by the application MIC tests.

A further embodiment of the invention provides for the contacting step of the methods to further comprise contacting an in-dwelling device in a patient. In-dwelling devices include, but are not limited to, surgical implants, prosthetic devices and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters.

A gemifloxacin compound or composition of the invention may be administered by injection to achieve a systemic effect against relevant bacteria, preferably a maxillary sinus pathogenic bacteria, shortly before insertion of an in-dwelling device. Treatment may be continued after surgery during the in-body time of the device. In addition, the composition could also be used to broaden perioperative cover for any surgical technique to prevent bacterial wound infections caused by or related to maxillary sinus pathogenic bacteria.

In addition to the therapy described above, a gemifloxacin compound or composition used in the methods of this invention may be used generally as a wound treatment agent to prevent adhesion of bacteria to matrix proteins, particularly maxillary sinus pathogenic bacteria, exposed in wound tissue and for prophylactic use in dental treatment as an alternative to, or in conjunction with, antibiotic prophylaxis.

Alternatively, a gemifloxacin compound or composition of the invention may be used to bathe an indwelling device immediately before insertion. The active agent will preferably be present at a concentration of 1μg/ml to 10mg/ml for bathing of wounds or indwelling devices.

Also provided by the invention is a method of treating or preventing a bacterial infection by maxillary sinus pathogenic bacteria comprising the step of administering an antibacterially effective amount of a composition comprising a gemifloxacin compound to a mammal, preferably a human, suspected of having or being at risk of having an infection with maxillary sinus pathogenic bacteria.

While a preferred object of the invention provides a method wherein said maxillary sinus pathogenic bacteria is selected from the group consisting of: a bacterial strain isolated from acute or chronic maxillary sinusitis; a maxillary sinus isolate of Staphylococcus aureus, Streptococcus pneumoniae, Haemophilus spp., Moraxella catarrhalis, an anaerobic strain or non-fermentative Gram negative bacilli, Neisseria meningitidis, β-haemolytic Streptococcus, Haemophilus influenzae, an Enterobacteriaceae, a non-fermentative Gram negative bacilli, Streptococcus pneumoniae, Streptococcus pyogenes, a methicillin-resistant Staphylococcus spp., Legionella pneumophila, Mycoplasma spp. and Chiamydia spp., Haemophilus influenzae, Haemophilus parainfluenzae, Peptostreptococcus, Bacteroides spp., and Bacteroides urealyticus. Other maxillary sinus pathogenic bacteria may also be included in the methods. The skilled artisan may identify these organisms as provided herein as well as using other methods known in the art, e.g. MIC tests.

Preferred embodiments of the invention include, among other things, methods wherein said composition comprises gemifloxacin, or a pharmaceutically acceptable derivative thereof.

### EXAMPLES

The present invention is further described by the following examples. The examples are provided solely to illustrate the invention by reference to specific embodiments. This exemplification's, while illustrating certain specific aspects of the invention, do not portray the limitations or circumscribe the scope of the disclosed invention.

All examples were carried out using standard techniques, which are well known and routine to those of skill in the art, except where otherwise described in detail.

All parts or amounts set out in the following examples are by weight, unless otherwise specified.

### Example 1

### Bacterial Strains

Test strains were obtained from recent maxillary sinus aspiration. Identification of organisms was by standard methods (see, for example, Murray, P. R., et al. Manual of Clinical Microbiology. 6th ed. American Society of Microbiology 1995: 282-620).

### Example 2

### Antimicrobial Activity Testing

Antimicrobial activity was tested against 250 selected isolates (Table 1). Emphasis was placed on testing commonly isolated sinusitis organisms or organisms that have demonstrated resistance to common oral therapy.

### Example 3

### Susceptibility Testing

The agar dilution method using replicate plating of the organisms onto a series of agar plates of increasing concentrations was used (see, for example, National Committee for Clinical Laboratory Standards. Methods for antimicrobial susceptibility tests for bacteria that growth aerobically. Approved standards M 7-A4. National Committee for Laboratory Standards, Villanova, Pa., 1997).

MICs were determined by using doubling dilutions of between 0.02-256 mg/L with an inoculum of 10⁴ CFU in area of 5-8 mm.

Mueller-Hinton agar was used for routine susceptibility testing of aerobic and facultative anaerobic bacteria and was supplemented with 5% defibrinated sheep blood for testing those organisms that do not grow on the unsupplemented medium. Haemophilus Test Medium was used for Haemophilus spp. and Wilkins-Chalgren agar was used for anaerobes. After incubation at 35° C. for 24 h in an aerobic atmosphere for aerobes or facultative anaerobes, in 5-7% CO₂ for Haemophilus and in an anaerobic atmosphere for anaerobes, the MIC was determined as the lowest concentration of antimicrobial that completely inhibited growth.

Each reference cited herein is hereby incorporated by reference in its entirety. Moreover, each patent application to which this application claims priority is hereby incorporated by reference in its entirety.

What is claimed is:

1. A method for modulating metabolism of maxillary sinus pathogenic bacteria comprising the step of contacting maxillary sinus pathogenic bacteria with an antibacterially effective amount of a composition comprising a gemifloxacin compound, or antibacterially effective derivatives thereof.

2. The method of claim 1 wherein said maxillary sinus pathogenic bacteria is selected from the group consisting of:
a bacterial strain isolated from acute or chronic maxillary sinusitis; and
a maxillary sinus isolate of S. aureus, S. pneumoniae, Haemophilus spp., M. catarrhalis, and anaerobic strain or non-fermentative Gram negative bacilli, Neisseria meningitidis and β-haemolytic Streptococcus.

3. A method of treating or preventing a bacterial infection by maxillary sinus pathogenic bacteria comprising the step of administering an antibacterially effective amount of a composition comprising a gemifloxacin compound to a mammal suspected of having or being at risk of having an infection with maxillary sinus pathogenic bacteria.

4. The method of claim 3 wherein said maxillary sinus pathogenic bacteria is selected from the group consisting of:
a bacterial strain isolated from acute or chronic maxillary sinusitis; and
a maxillary sinus isolate of S. aureus, S. pneumoniae, Haemophilus spp., M. catarrhalis, and anaerobic strain or non-fermentative Gram negative bacilli, Neisseria meningitidis and β-haemolytic Streptococcus.

5. The method of claim 1 wherein said modulating metabolism is inhibiting growth of said bacteria.

6. The method of claim 1 wherein said modulating metabolism is killing said bacteria.

7. The method of claim 1 wherein said contacting said bacteria comprises the further step of introducing said composition into a mammal.

8. The method of claim 3 wherein said mammal is a human.

9. The method of claim 7 wherein said mammal is a human.

10. The method of claim 1 wherein said bacteria is selected from the group consisting of: a bacterial strain isolated from acute or chronic maxillary sinusitis; a maxillary sinus isolate of Staphylococcus aureus, Streptococcus pneumoniae, Haemophilus spp., Moraxella catarrhalis, an anaerobic strain or non-fermentative Gram negative bacilli, Neisseria meningitidis, β-haemolytic Streptococcus, Haemophilus influenzae, an Enterobacteriaceae, a non-fermentative Gram negative bacilli, Streptococcus pneumoniae, Streptococcus pyogenes, a methicillin-resistant Staphylococcus spp., Legionella pneumophila, Mycoplasma spp. and Chlamydia spp., Haemophilus influenzae, Haemophilus parainfluenzae, Peptostreptococcus, Bacteroides spp., and Bacteroides urealyticus.

11. The method of claim 1 wherein said bacteria is selected from the group consisting of: a bacterial strain isolated from acute or chronic maxillary sinusitis; a maxillary sinus isolate of Staphylococcus aureus, Streptococcus pneumoniae, Haemophilus spp., Moraxella catarrhalis, an anaerobic strain or non-fermentative Gram negative bacilli, Neisseria meningitidis, β-haemolytic Streptococcus, Haemophilus influenzae, an Enterobacteriaceae, a non-fermentative Gram negative bacilli, Streptococcus pneumoniae, Streptococcus pyogenes, a methicillin-resistant Staphylococcus spp., Legionella pneumophila, Mycoplasma spp. and Chlamydia spp., Haemophilus influenzae, Haemophilus parainfluenzae, Peptostreptococcus, Bacteroides spp., and Bacteroides urealyticus.
